(19) United States
(12) Patent Application Publication
(10) Pub. No.: US 2004/0245164 A1
(43) Pub. Date: Dec. 9, 2004

(54) Fluid purification system

(76) Inventors: Harvey R. Sellner, Newtown, CT (US); Robert J. Vetter, Danbury, CT (US)

Correspondence Address: MELVIN I. STOLTZ, ESQ., 51 CHERRY STREET, MILFORD, CT, 06460, US

(21) Appl. No.: 10/482,501

(22) PCT Filed: Jun. 25, 2002
(86) PCT No.: PCT/US02/20207

Related U.S. Application Data
(60) Provisional application No. 60/302,443, filed on Jul. 2, 2001.

(51) Int. Cl. B01D 33/00

(52) U.S. Cl. 210/323.2; 210/192; 210/321.6; 250/435; 422/186.3

(57) ABSTRACT

By providing a fluid flow chamber which is preferably constructed in an elongated, cylindrical shape and incorporates an elongated cylindrical sleeve in which an ultraviolet radiation producing lamp is positioned along the central axis thereof, with the outer surface of the sleeve covered with a unique coating layer, a highly efficient and low cost fluid purification system has been achieved which employs ultraviolet radiation. The unique coating material of the present invention allows the ultraviolet radiation to pass therethrough substantially unimpeded, while also virtually eliminating any buildup of light blocking deposits from the fluid on the surface thereof. As a result, the purification system of the present invention is capable of providing the desired high radiation level exposures for substantially extended periods of time without requiring repeated maintenance. Furthermore, an inlet port is formed at one end of the elongated chamber with an outlet port formed at the opposed end, whereby the fluid to be purified flows through the elongated chamber, while simultaneously being exposed to the ultraviolet radiation, while the buildup of radiation blocking impurities is eliminated.

### TECHNICAL FIELD

This invention relates to fluid purification systems and, more particularly, to fluid purification systems employing the exposure of the fluid to ultraviolet radiation.

### BACKGROUND ART

Substantial effort has been expended over the many years in developing treatment or purification systems which seek to provide high-quality, potable drinking water for domestic use and/or commercial use. In spite of the substantial effort that has been expended in this area and the user's desire for substantially improving the quality of the water being consumed, no system has been developed which is capable of satisfying all of the industry and consumer demands and requirements.

Many prior art systems that have been developed employ filters, such as activated carbon filters or reverse osmosis, for attempting to improve the quality of the water passing therethrough. However, due to the inherent problems associated with such filters and reverse osmoses, these prior art systems have been incapable of providing the high-quality drinking water being sought by consumers.

In addition, exposure of water to ultraviolet radiation has also been employed in numerous prior art constructions, due to the inherent capability ultraviolet radiation possesses for killing microorganisms and bacteria carried by the water. However, these prior art constructions have failed to satisfy the needs and demands of the consumer, due to high cost of construction found in many of these prior art systems, as well as the inability of other, less-expensive systems to be capable of long-term effective use, without experiencing system degradation.

One major problem that has consistently plagued prior art products is the continuous buildup of particulate matters or impurities on any surface coming in contact with the fluid. As particulates continue to be deposited and build up on these surfaces, the transmission of the ultraviolet radiation is reduced and the overall efficacy of the system is compromised.

The only way in which this problem is presently addressed is manual cleaning of the affected surfaces. However, this cleaning is difficult, time-consuming, and requires constant vigilance. In addition, the ultraviolet radiation level is continuously reduced as the buildup of the particles continues until removed.

Therefore, it is a principal object of the present invention to provide a water purification system employing ultraviolet radiation which is capable of being manufactured inexpensively, achieving a cost effective and highly competitive system.

Another object of the present invention is to provide a water purification system having the characteristic features described above which is specifically constructed for enabling long-term use without experiencing degradation or substantial reduction in the efficacy of the ultraviolet radiation.

Another object of the present invention is to provide a water purification system having the characteristic features described above which employs a minimum of components, is small and compact for use and installation in any desired location.

Another object of the present invention is to provide a water purification system having the characteristic features described above which is constructed for ease of maintenance whenever needed, as well as for protecting the water supply from contamination by airborne bacteria.

Other and more specific objects will in part be obvious and will in part appear hereinafter.

### SUMMARY OF THE INVENTION

By employing the present invention, all of the difficulties and drawbacks found in the prior art have been overcome and a highly efficient and low cost, fluid purification system has been achieved which employs ultraviolet radiation. In accordance with the present invention, a fluid flow chamber is provided which is preferably constructed in an elongated, cylindrical shape incorporating an elongated, ultraviolet radiation producing lamp positioned along the central axis thereof. Furthermore, an inlet port is formed at one end of the elongated chamber with an outlet port formed at the opposed end. In this way, the fluid to be purified flows through the elongated chamber, while simultaneously being exposed to the ultraviolet radiation.

In order to assure complete elimination of the prior art difficulties encountered with ultraviolet radiation reduction over time, due to the fouling of transmission surfaces by deposits in the water, the surfaces through which the ultraviolet radiation passes are treated with a unique coating material. In accordance with this invention, the coating material allows the ultraviolet radiation to pass therethrough substantially unimpeded, while also virtually eliminating any build up of light blocking deposits on the surfaces. As a result, the purification system of the present invention is capable of providing the desired high radiation level exposures for substantially extended periods of time, without requiring special maintenance. In this way, many of the difficulties and drawbacks of the prior art are eliminated.

In order to provide a highly effective, cost-efficient system which is easily maintained while also assuring a completely sealed fluid flow path, the preferred embodiment of the present invention incorporates an elongated, cylindrically shaped sleeve or tube member mounted along the central axis of the cylindrically shaped chamber and constructed for receiving and retaining the ultraviolet lamp therein. By employing this elongated sleeve or tube member, a completely sealed fluid flow chamber is realized. In addition, with the tube member sealingly affixed to the terminating ends of the cylindrical chamber, a completely sealed, leak-free fluid flow path is established, defined by the outer surface of the sleeve or tube member and the inner surface of the chamber.

By employing this construction, the ultraviolet lamp can be quickly and easily telescopically inserted into the sleeve or tube member for being retained therein, as well as being quickly and easily removed and replaced with a new ultraviolet lamp when such maintenance is needed. As a result, a completely sealed fluid flow path is provided along with a construction enabling rapid removal and replacement of the ultraviolet lamp whenever required.

In this preferred embodiment of the present invention, the elongated, cylindrically shaped sleeve or tube member is constructed from quartz material in order to enable the ultraviolet radiation to efficiently and effectively pass therethrough, directly into the fluid flowing through the chamber. As is well known in the art, quartz provides an optimum material for enabling the ultraviolet radiation to be transmitted therethrough, with minimal degradation or loss. As a result, the elongated, cylindrically shaped sleeve or tube member is preferably constructed from quartz in order to attain the inherent benefits from this material.

In addition, in the preferred embodiment of the present invention, the sleeve/tube member is treated on its outer surface with a unique coating material of the present invention. Since the fluid, typically water, passing through the fluid flow chamber comes in direct contact with the outer surface of sleeve/tube member, the incorporation of the unique coating material on the outer surface of the sleeve/tube member provides assurance that impurities in the fluid or water will not buildup or adhere to the outer surface of the sleeve/tube member. As a result, any unwanted degradation of the transmission capabilities of the sleeve/tube member is prevented.

Preferably, the inside surface of the cylindrically shaped chamber is coated with highly reflective material, in order to optimize the concentration of the ultraviolet radiation into the fluid flowing through the chamber. By employing the highly reflective material, the ultraviolet radiation passing through the sleeve/tube member and through the fluid flowing through the chamber is reflected off of the inside wall of the chamber back into the fluid. As a result, optimum exposure of the fluid to the ultraviolet radiation is attained.

In the present invention, in order to prevent any unwanted buildup of light blocking impurities on the inside surface of the tube member, the inside surface of the tube member is also treated with the coating material employed on the sleeve/tube member. In this way, optimum ultraviolet radiation transmission is assured and unwanted degradation of the inside surface of the chamber is prevented.

In accordance with the present invention, any coating material which is capable of transmitting ultraviolet radiation with virtually no degradation of the signal, while also being capable of resisting water impurities from adhering to the coating material can be employed in the present invention. Although various such materials are available, it has been found that coating is formed from fluoropolymers, preferably selected from the Teflon family of products, providing optimum results. Alternatively, if desired, some silicone based materials which are transmissive to ultraviolet radiation and resist the buildup of impurities from the fluid, can also be employed.

By employing the present invention, periodic cleaning or replacement of components is eliminated and a long-lasting, highly efficient, cost-effective system is realized. Furthermore, if desired, the water purification system of the present invention may also be employed with additional filtration products, such as carbon filters or reverse osmosis units, in order to present additional impurities from passing therethrough. If employed, the fluid purification system may contain one more additional chambers to prevent reverse contamination of the additional equipment.

The invention accordingly comprises an article of manufacture possessing the features, properties, and the relation of elements which will be exemplified in the article hereinafter described, and the scope of the invention will be indicated in the claims.

### THE DRAWINGS

For a fuller understanding of the nature and objects of the invention, reference should be had to the following detailed description taken in connection with the accompanying drawings, in which:

FIG. 1 is a cross-sectional side elevation view of the water purification system of the present invention; and

FIG. 2 is a cross-sectional end view of the water purification system of the present invention taken along line 2-2 of FIG. 1.

### DETAILED DISCLOSURE

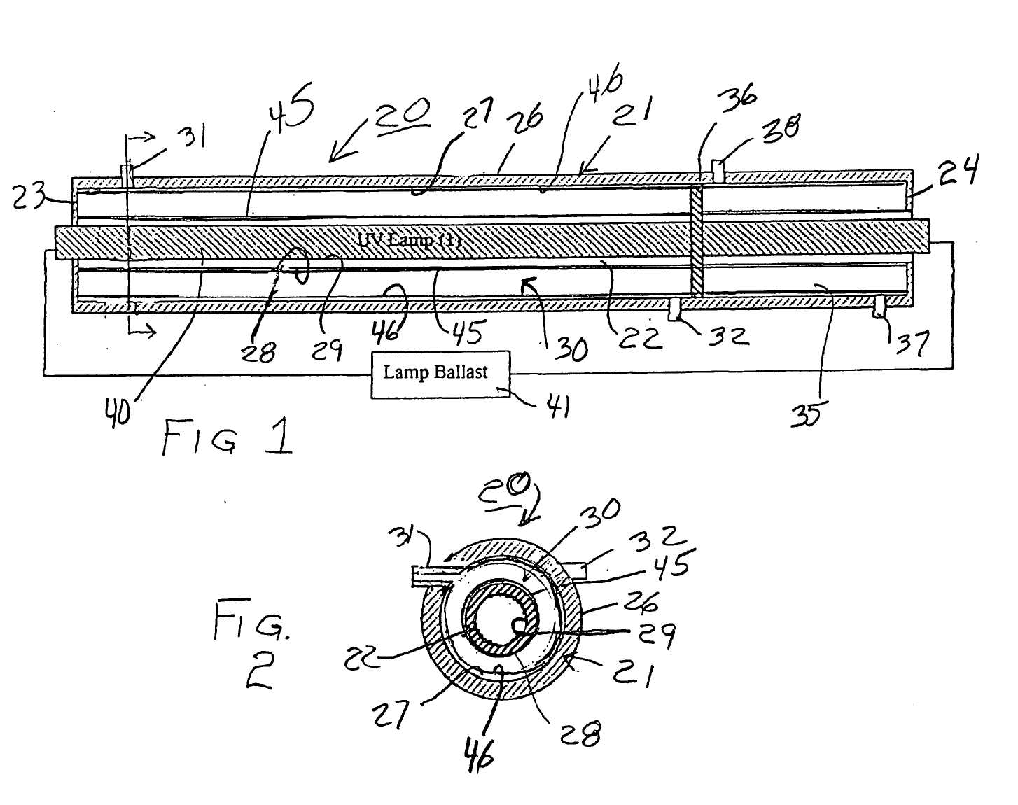

By referring to FIGS. 1 and 2, along with the following detailed discussion, the construction and operation of the fluid purification system of the present invention can best be understood. In this disclosure, the preferred embodiment of the present invention is fully detailed. However, alternate embodiments can be made without departing from the scope of the present invention. Consequently, it is to be understood that the disclosure contained herein is provided for exemplary purposes only and is not intended as a limitation of the present invention.

Furthermore, it is also to be understood that the present invention can be employed for purifying any desired fluid. Although water is most commonly used as the fluid to be purified, the present invention is not limited to water and any other fluids can be purified, including wine, cider, juices, and gases. Furthermore, although the present invention is detailed in regards to its applicability for use with water, this term shall be considered equivalent to and encompass any desired fluid.

In FIGS. 1 and 2, the preferred embodiment and construction of fluid sterilization system 20 is depicted. As shown, in this embodiment, fluid sterilization system 20 comprises an elongated chamber or housing 21, ultraviolet radiation transparent sleeve member 22, and end walls 23 and 24. In this preferred construction, chamber/housing 21 comprises an elongated, hollow, cylindrical shape defined by outer wall 26 and inner wall 27. In addition, end walls 23 and 24 are affixed to the terminating ends thereof, effectively sealing chamber/housing 21 and establishing a leak-free interior zone.

Furthermore, ultraviolet radiation transparent sleeve member 22 also comprises an elongated, hollow cylindrical shape, defined by outer wall 28 and inner wall 29. In the preferred embodiment, sleeve member 22 is mounted in chamber/housing 21 with the central axis of sleeve member 22 coinciding with the central axis of chamber/housing 21. In addition, the terminating ends of sleeve member 22 are sealingly affixed to end walls 23 and 24, thereby preventing leakage of any fluid flowing within chamber/housing 21.

By mounting sleeve member 22 along the central axis of elongated chamber/housing 21, fluid flow zone 30 is established, defined by inner wall 27 of chamber/housing 21 and outer wall 28 of sleeve member 22. In addition, fluid sterilization system 20 also comprises inlet portal 31 and outlet portal 32 mounted to elongated chamber/housing 21 for delivering the fluid directly into fluid flow zone 30, for sterilization therein, and withdrawal of the sterilized fluid from zone 30.

By positioning inlet portal 31 at one end of fluid flow zone 30 and positioning outlet portal 32 at the opposed end of fluid flow zone 30, optimum exposure of the fluid to the ultraviolet radiation is attained. Furthermore, the residence time of the fluid passing through zone 30 is maximized. In this regard, either portal may be employed as the inlet or the outlet.

It has been found that in the overall cylindrical construction of chamber/housing 21, along with inlet portal 31 and outlet portal 32 being mounted substantially perpendicular to the wall defining chamber/housing 21, the fluid entering portal 31 flows substantially perpendicular to sleeve member 22, progressing through the length of chamber/housing 21 in a substantially circular motion, continuously advancing towards outlet portal 32. If desired, optional baffle means may be incorporated in fluid flow zone 30 for uniformizing the flow of fluid therethrough as well as creating turbulence in order to further optimize the residence time of the fluid in zone 30.

If desired, the entire length of chamber/housing 21 can be employed for purifying the fluid passing therethrough. However, if desired, chamber/housing 21 may be divided into two or more separate and independent flow zones. In FIG. 1, two flow zones are depicted, with fluid flow zone 30 representing the primary sterilization section, and fluid flow zone 35 being formed, representing a separate and independent, secondary fluid flow chamber.

In this construction, secondary fluid flow zone 35 is established by mounting partition member 36 in housing/chamber 21, with partition member 36 peripherally surrounding sleeve member 22 and extending therefrom into secure engagement with inside surface 27 of chamber/housing 21. In addition, secondary inlet portal 37 is mounted at one end of fluid flow zone 35 with secondary outlet portal 38 being mounted at the opposed end of fluid flow zone 35. In this way, a secondary fluid flow zone is established, with the same flow advantages being provided for sterilization of the fluid, as the fluid flows into zone 35 through portal 37 and out of zone 35 through portal 38.

Depending on the overall fluid purification system being employed, primary and secondary fluid flow zones are desirable. In particular, if additional purification systems such as carbon filters and reverse osmosis equipment are employed, the incorporation of secondary fluid flow zone 35 is desirable for preventing reverse contamination of these additional purification components.

If employed, these additional purification systems would be connected to the fluid flow path after outlet portal 32, with the completely purified fluid reaching the consumer after passage through the additional equipment. However, it has been found that reverse contamination or back contamination of these additional purification components can be experienced due to airborne microbes or other contaminants entering the exit port of the added components. As a result, secondary flow chamber 35 should be employed in a construction of this nature, in order to assure that this secondary contamination is eliminated and any contaminants added to the water are purified by passage through secondary flow zone 35.

Since the principal purification of any fluid is achieved in fluid flow zone 30, fluid flow zone 30 is constructed with a length substantially greater than the length of any secondary flow zone being employed. In this regard, the overall axial length of fluid flow zone 30 is specifically designed for assuring that the fluid contaminants are killed by the ultraviolet radiation as the fluid passes through flow zone 30 at the known flow rate being employed.

By employing these factors, assurance is provided that optimum efficiency and fluid purification is realized. Furthermore, bacteria and/or organism growth in any carbon filter resulting from back contamination is an extremely slow process since organisms need to migrate against the direction of the fluid flow in order to infiltrate the system. Therefore, secondary fluid flow zone 35 can be extremely short since the organism residence time is quite long.

As shown in FIG. 1, germicidal, ultraviolet radiation producing lamp 40 is mounted in sleeve member 22 for producing the desired ultraviolet radiation which purifies the fluid passing through flow zones 30 and 35. In the preferred construction, ultraviolet radiation producing lamp 40 is controlled by lamp ballast 41 and is mounted in sleeve member 22 for ease of insertion and removal. In this way, whenever lamp 40 needs to be replaced, such replacement can be achieved quickly and easily, by merely telescopically withdrawing lamp 40 from sleeve member 22 and telescopically inserting the replacement lamp therein.

In accordance with the present convention, sleeve member 22 is formed from any desired material which is virtually transparent to ultraviolet radiation. Although various materials meeting this criteria may be employed, it has been found that quartz provides optimum results and represents the preferred material from which sleeve member 22 is formed.

In addition, as discussed above, as the fluid being purified travels through fluid flow zone 30, impurities which are contained and/or suspended in the fluid, are deposited on outer surface 28 of sleeve member 22. As the flow of the fluid through zone 30 continues, additional impurity deposits are formed on surface 28, causing an ever-increasing buildup of unwanted material.

In view of the fact that impurity buildup on surface 28 of sleeve member 22 prevents the ultraviolet radiation from passing through those areas where impurity buildup exists, the efficacy of the ultraviolet radiation reaching the fluid is substantially reduced and impaired. As has been found in conventional prior art constructions, this continual buildup of impurities requires either the complete removal and replacement of sleeve member 22 or constant, repeated cleaning thereof.

In order to completely eliminate the prior art difficulty encountered with impurity buildup on sleeve member 22, outer surface 28 of sleeve member 22 incorporates coating layer 45 affixed thereto. In the preferred embodiment, coating layer 45 is specifically selected from those materials which are virtually transparent to ultraviolet radiation while also incorporating the inherent quality of resisting impurity affixation or buildup thereon.

As a result, ultraviolet radiation transmission through coating layer 45 is virtually unaffected, while any buildup of impurities on the surface of coating layer 45 is prevented. In this way, optimum efficiency and performance of ultraviolet radiation transmission is realized.

In order to effectively cover outer surface 28 of sleeve member 22 with coating layer 45, it has been found coating layer 45 is applied to sleeve member 22 for peripherally surroundings sleeve member 22 in its entirety, virtually encapsulating sleeve member 22 in a continuous, unitary, fully integrated coating layer construction. In this way, any possibility of impurity buildup is prevented and optimum efficacy of coating layer 45 is realized.

Although coating layer 45 may be selected from various materials meeting the criteria defined above, it has been found that coating layer 45 preferably comprises one selected from the group consisting of polytetrafluoroethylenes, fluoropolymer resins, fluorinated ethylene propylene resins and perfluoroalkoxy copolymer resins. In this regard, the preferred material comprises one selected from the group consisting of Teflon and the Teflon family of products, all of which are trademarks of The DuPont Company. In addition, some silicone materials, which are transmissive to ultraviolet radiation and are resistant to the buildup of impurities from fluids, can also be employed.

In order to further enhance the efficacy of the present invention, including the ability of sterilization system 20 to optimize ultraviolet radiation exposure to the fluid passing through fluid flow zones 30 and 35, chamber/housing 21 incorporates an ultraviolet radiation reflective surface or surface coating applied to inside surface 27 thereof. Although any desired material can be employed, chamber/housing 21 is preferably formed from aluminum, with inside surface 27 being polished for optimum reflectivity or coated with a suitable coating material, such as chrome, which provides the desired ultraviolet radiation reflectivity.

In addition, in the preferred embodiment, inside surface 27 of chamber/housing 21 also incorporates coating layer 46 affixed thereto. In this regard, coating layer 46 comprises a material similar to the material detailed above for coating layer 45. By affixing coating layer 46 to inside surface 27 of chamber/housing 21, buildup of impurities on inside surface 27 is virtually eliminated, thereby providing long term use of fluid sterilization system 20 without requiring repeated cleaning of inside surface 27.

In carrying out the teaching of the present invention, coating layer 45 can be applied to outer surface 28 of sleeve member 22 in any desired thickness. However, it has been found that in the preferred construction, coating layer 45 comprises an overall thickness ranging between about 0.001 inches and 0.005 inches. Similarly, any desired thickness can be employed for coating layer 46 as applied to inside surface 27 of chamber/housing 21. However, in the preferred construction, coating layer 46 comprises a thickness ranging between about 0.001 inches and 0.005 inches.

In addition, depending upon the quantity of fluid to be purified, fluid sterilization system 20 may be constructed with any desired overall dimensions. However, for most applications, wherein a flow rate ranging between about one gallon per minute and two gallons per minute is desired, optimum results have been attained wherein chamber/housing 21 comprises an inside diameter ranging between about 1 inch and 1.5 inches, with fluid flow zone 30 and 35 comprising an overall width ranging between about 0.25 inches and 0.8 inches. In this way, a highly compact, easily used and efficient system is realized.

Other features that may be incorporated into fluid sterilization system 20, if desired, is a sleep mode or a low-power mode, which automatically turns off ultraviolet lamp 40, or reduces the intensity of lamp 40, whenever fluid is not flowing through chamber/housing 21. In this way, the usable life of ultraviolet lamp 40 is optimized. Furthermore, ultraviolet level monitors may also be incorporated into fluid sterilization system 20 in order to assure the user that the desired ultraviolet radiation dosage is being attained.

It will thus be seen that the objects set forth above, among those made apparent from the preceding description, are efficiently attained and, since certain changes may be made in the above construction without departing from the scope of the invention, it is intended that all matter contained in the above description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween.

Having described our invention, what we claim as new and desire to secure by Letters Patent is:

1. A fluid purification system comprising:
A. an elongated housing incorporating a sealed interior defining a fluid transfer zone;
B. a first portal mounted to the housing in communicating relationship with the fluid transfer zone for delivering a fluid into said zone;
C. a second portal mounted to the housing in communicating relationship with the fluid transfer zone for withdrawing fluid from said zone;
D. an elongated, hollow, sleeve member
a. mounted in said fluid transfer zone,
b. comprising an outside surface and an inside surface which defines an interior receiving cavity, and
c. constructed for receiving and supportingly maintaining an ultraviolet radiation-producing lamp in said receiving cavity thereof;
E. an ultraviolet radiation-producing lamp removably mountable in the receiving cavity of the sleeve member and constructed for delivering ultraviolet radiation into said fluid transfer zone; and
F. a coating layer
a. formed on the outside surface of said sleeve member in peripherally surrounding, substantially encapsulating relationship therewith, and
b. comprising material which is virtually transparent to ultraviolet radiation and is resistant to any buildup of particulate matter suspended in said fluid;
whereby a fluid purification system is attained which eliminates repeated cleaning of the sleeve member for removal of unwanted particle buildup thereon.

2. The fluid purification system defined in claim 1, wherein said sleeve member is further defined as being sealed within the fluid transfer zone for preventing leakage of fluid from said zone.

3. The fluid purification system defined in claim 2, wherein said elongated housing is further defined as comprising a hollow cylindrical shape incorporating an outside surface, an inside surface, and a first end wall and a second end wall both sealingly engaged therewith for defining a leak-free fluid transfer zone.

4. The fluid purification system defined in claim 3, wherein said elongated, hollow, sleeve member is further defined as being mounted in said housing along the central axis thereof and for cooperating with the housing to define the fluid transfer zone as the area between the inside surface of the housing and the outside surface of the sleeve member.

5. The fluid purification system defined in claim 4, wherein said inlet portal is further defined as being mounted adjacent said first end wall and said outlet portal is further defined as being mounted adjacent the second end wall.

6. The fluid purification system defined in claim 5, when said inlet portal is further defined as being positioned in said housing for delivering the fluid into the fluid transfer zone in a manner which maximizes the residence time of the fluid in said zone.

7. The fluid purification system defined in claim 6, wherein said elongated, hollow sleeve member is further defined as being formed from quartz.

8. The fluid purification system defined in claim 7, wherein said coating layer is further defined as comprising one selected from the group consisting of polytetrafluoroethylenes, fluoropolymer resins, fluorinated ethylene propylene resins and perfluoroalkoxy copolymer resins.

9. The fluid purification system defined in claim 8, wherein the inside surface of said housing is further defined as being reflective to ultraviolet radiation.

10. The fluid purification system defined in claim 9, wherein said reflective inside surface of said housing is further defined as comprising reflective material formed the thereon.

11. The fluid purification system defined in claim 10, wherein the inside surface of said housing is further defined as comprising a coating layer consisting of material which is virtually transparent to ultraviolet radiation and is resistant to any buildup of particulate matter suspended in said fluid.

12. The fluid purification system defined in claim 1, wherein said system further comprises additional filtration equipment associated therewith, said filtration equipment comprising one selected from the group consisting of filters and reverse osmosis products.

13. The fluid purification system defined in claim 12, wherein said elongated housing comprises a partitioning wall formed therein effectively dividing said housing into said a separate and independent primary fluid transfer zone and a separate and independent secondary fluid transfer zone.

14. The fluid purification system defined in claim 13, wherein said housing further comprises a separate inlet portal for delivering fluid into said secondary fluid transfer zone and a separate outlet portal for withdrawing fluid from said secondary fluid transfer zone and the additional filtration equipment is connected between the primary fluid transfer zone and the secondary fluid transfer zone, thereby preventing reverse contamination.
